(19) United States
(12) Patent Application Publication
(10) Pub. No.: US 2024/0158639 A1
(43) Pub. Date: May 16, 2024

(54) PROCESS FOR PREPARING INDOCYANINE GREEN

(71) Applicant: ICROM Srl, Concorezzo (MB) (IT)

(72) Inventors: Daniele De Zani, Concorezzo (MB) (IT); Simone Parma, Concorezzo (MB) (IT) (IT)

(73) Assignee: ICROM Srl, Concorezzo (MB) (IT)

(21) Appl. No.: 18/546,342

(22) PCT Filed: Mar. 21, 2022
(86) PCT No.: PCT/IB2022/052551

§ 371 Date: Aug. 14, 2023

(30) Foreign Application Priority Data
Mar. 22, 2021 (IT) 102021000006794

(51) Int. Cl. C09B 23/08; C07D 209/60; C09B 67/42; C09B 67/54; G01N 30/74; G01N 30/88

(52) U.S. Cl. CPC C09B 23/083; C07D 209/60; C09B 67/0092; C09B 67/0096; G01N 30/74; G01N 30/88; G01N 2030/027

(57) ABSTRACT

The present invention relates to a process for the preparation, also on an industrial scale, of indocyanine green of formula (I) (ICG, 1 H-benz[e]indole, 2-[7-[1,3-dihydro-1,1-dimethyl-3-(4-sulfobutyl)-2H-benz[e]indol-2-ylidene]-1,3,5-heptatrienyl]-1,1-di-methyl-3-(4-sulfobutyl) hydroxide, inner salt, sodium salt, CAS RN 3599-32-4) with a total impurity content <0.5% and % and single impurity <0.10%, MeOH free, purity determined by a new analytical method HPLC at the wavelength of 254 nm, and the related composition with stable NaI, which is water soluble and has NaI content <2.5%.

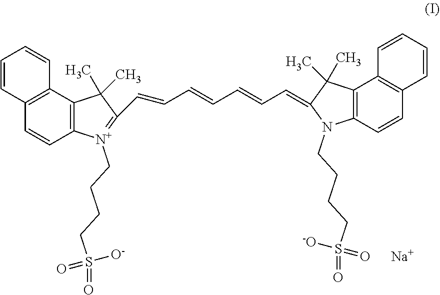

The present invention relates to a process for the preparation, also on an industrial scale, of indocyanine green of formula (I) (ICG, 1H-benz[e]indole, 2-[7-[1,3-dihydro-1,1-dimethyl-3-(4-sulfobutyl)-2H-benz[e]indol-2-ylidene]-1,3,5-heptatrienyl]-1,1-dimethyl-3-(4-sulfobutyl) hydroxide, inner salt, sodium salt, CAS RN 3599-32-4) with a total impurity content ≤0.5% and % and single impurity ≤0.10%, MeOH free, purity determined by a new analytical method HPLC at the wavelength of 254 nm, and the related composition with stable NaI, which is water soluble and has NaI content ≤2.5%.

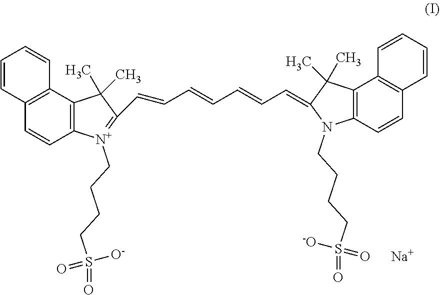

Indocyanine green is a fluorescent dye used in medicine as a contrast agent (e.g. for photometric diagnostics of liver function and fluorescence angiography) in heart, circulatory, liver and ophthalmic conditions. It is administered intravenously and, according to liver performance, is eliminated by the body with a half-life of about 3-4 minutes. The sodium salt of indocyanine green is normally available in powder form and can be dissolved in various solvents. 5% sodium iodide is usually added (<5% according to the batch) to guarantee better solubility. The sterile freeze-dried product of a water-indocyanine green solution is approved in many European countries and in the USA as a diagnostic for intravenous use. Various processes are known for the preparation of indocyanine green, including the patents U.S. Pat. No. 2,895,955 (filed in 1959) and U.S. Pat. No. 10,287,436 (filed in 2016) and the recent American patent US 2019/0337896.

The following synthesis diagram also reports these two synthetic approaches which are known in literature and reported in the mentioned references:

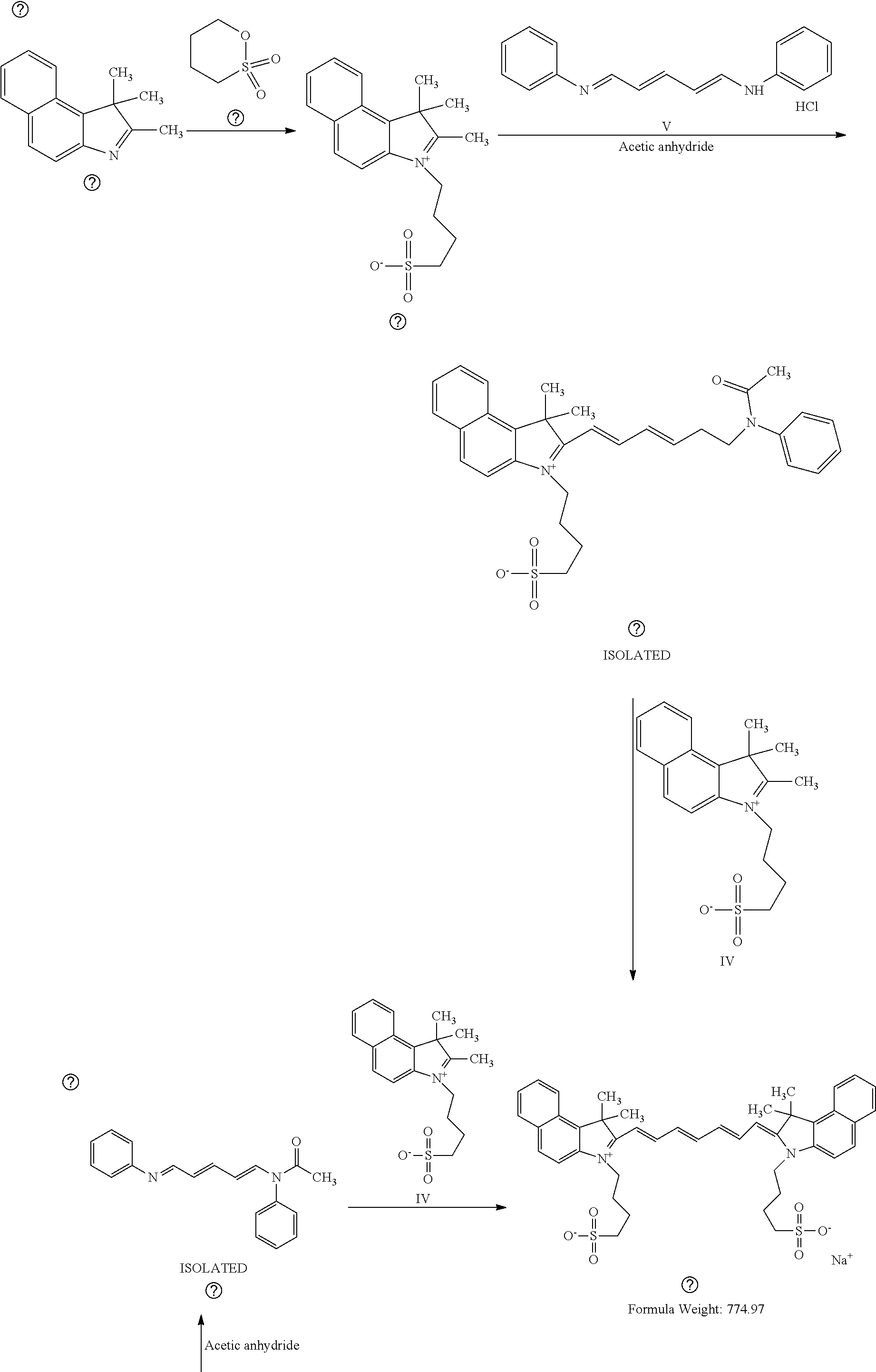

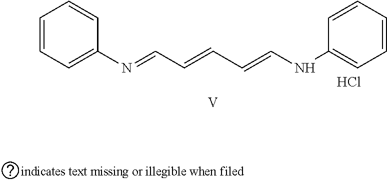

In U.S. Pat. No. 2,895,955 the compound of formula (I) is synthesized by the isolation of the intermediate (VI).

In U.S. Pat. No. 10,287,436 the compound of formula (I) is synthesized by pre-forming and isolating the intermediate (VII).

In US 2019/0337896 the compound of formula (I) is synthesized also here by passing through the isolation of the intermediate (VI).

Patent application US2019/0337896 also described the preparation of an amorphous form of the compound of formula (I) with purity greater than 99%. The application also describes the HPLC method used for determining such purity.

The Applicant prepared the compound of formula (I) following the indications present in the patent application, but found that the product obtainable by reproducing the examples of US2019/0337896 is not characterized by a purity ≥99% as declared in the same patent application, when analysed with the HPLC analytical method used in the present invention, as will be discussed in detail below.

The object of the present invention is to provide a new process for the preparation of the compound of formula (I) which is able to provide a final product characterized by a total impurity content ≤0.5% and a single impurity ≤0.1% and which can also be scalable on an industrial scale.

The process of the present invention also enables the composition comprising the compound of formula (I) and a lower NaI content (≤2.5%) to be obtained with respect to what is currently available on the market and, despite this, soluble in water up to 5 mg/ml and stable in the storage conditions commonly used for products currently on the market, i.e. protected from light and oxygen.

In fact, the products currently on the market have a higher NaI content (≤5%, in accordance with the American pharmacopoeia, USP).

The degree of impurities of the compound of formula (I) of the present invention is determined using two new HPLC methods that are characterized by the use of a different analysis wavelength with respect to the one previously applied, e.g. in patent application US2019/0337896.

In particular, by using the new HPLC analytical method with an analysis wavelength of 254 nm rather than 205 nm, the compound of formula (I) synthesized using the examples of US2019/0337896 was characterized by a much lower purity, i.e. 93%.

The process of the present invention is able to save on the synthetic steps thus making the process as a whole cheaper, avoiding both the isolation of the intermediate (VI) and the isolation of the intermediate (VII), thus differing from the mentioned state of the art.

It is further highlighted that the high degree of purity reached can be obtained thanks to the use of a special mixture of solvents and the special conditions used during the re-crystallization step and that such purity is actually reached despite the isolation of both (VI) and (VII) being avoided.

The present invention provides a new process for synthesis that comprises the following steps:
a. reacting the compound of formula (II) 1,1,2-trimethyl-1h-benzo[e]indole,

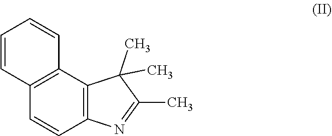 [SMILES: CC1=Nc2ccc3ccccc3c2C1(C)C]

with 1,4-butansultone of formula (III)

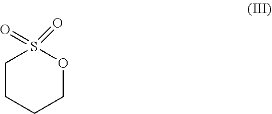

in an appropriate high boiling point solvent selected from anisol or xylene and in the absolute absence of methane to provide 4-(1,1,2-trimethyl-1H-benzo[e]indolyl-3-yl)butan-1-sulfonate of formula (IV), according to known methods;

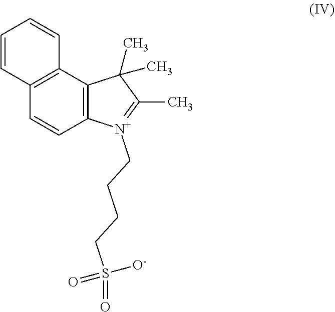 [SMILES: CC1=[N+](CCCCS(=O)(=O)[O-])c2ccc3ccccc3c2C1(C)C]

b. reacting the compound of formula (IV) with the compound formula (V),

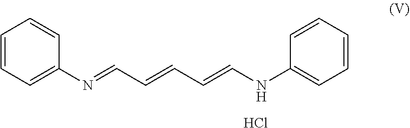 [SMILES: C(=C\C=N\c1ccccc1)/C=C/Nc1ccccc1 ; Cl]

benzenamine, N-[(2E, 4E)-5-(phenylamino)-2,4-pentadien-1-ylidene]-, hydrochloride (1:1) (known as GAD), in the presence acetic anhydride, sodium acetate and using a dipolar aprotic solvent to provide the final compound of formula (I), without isolating any intermediate.

Step a) is also well known in the references previously mentioned. The reaction can be performed at a temperature that depends on the high boiling point solvent used. US 2019/0337896 indicated the following aprotic solvents: hexane, cyclohexane, toluene, xylene, tetrahydrofuran, acetone, acetonitrile, 1,4-dioxane, diethyl ether, dichloromethane, ethyl acetate, N,N-dimethylformamide, methyl tert-butyl ether or the like, xylene and acetone.

The Applicant used xylene as a solvent at a temperature of around 130° C. Anisol can also be used with good results in terms of reaction speed, obtaining complete conversions in 7-8 hours at 140-150° C. instead of the usual 24 h necessary with xylene at 125-130° C. The intermediate compound of formula (IV) is isolated by precipitation adding acetone to the reaction mixture and is used as such, wet, without being recrystallized as described by US 2019/0337896.

The presence of methanol in this step was evaluated as a critical parameter. In fact, methanol can be used as a solvent for cleaning the reactors as it is good at dissolving all the materials used in this step, especially when the process is performed on an industrial scale. If, however, methanol remains inside the reactor, also in traces, it can react with the compound (II) to produce N-Methyl-Benzindole (impurity G), which in the following step will generate the impurity “Methyl-Indocyanine” (impurity H).

In a test in the pilot system (“demo batch” test) this impurity was in fact found and its hypothetical structure like that of “Methyl-Indocyanine” (impurity H) on the basis of the LC/MS study (LC/MS ESI⁺; [MH]⁺: 631,39). Such a structure was confirmed through total synthesis, as reported below in the Experimental Part.

The compound (IV) that generated it was in turn analysed through LC/MS revealing the presence of the impurity Methyl-Benzindole (impurity G), characterized by the expected m/z (LC/MS, ESI⁺: [M]⁺: 224 found). Also in this case, the structure was confirmed by total synthesis.

The absence of methanol, also as a residual solvent in the starting materials, guarantees that a product is obtained with the impurity H specifically, i.e. less than 0.10%. The evaluation of this impurity was then improved through the development of an HPLC method suitable for this purpose.

The process according to the present invention is hence characterized by the direct performance of step b), i.e. “one step”, without isolating any intermediate and without the need to purify either the intermediate (VI) or the intermediate (VII), as happened in synthesis already known in the state of the art and discussed previously.

Step b) is performed as already known by the condensation of the compound of formula (IV) with the compound of formula (V) in the presence of a solvent (acetonitrile), acetic anhydride and sodium acetate. The reaction is performed at a temperature comprised between 40-50° C. to form the crude compound of formula (I). The compound of formula (V) and the compound of formula (IV) are dissolved in acetonitrile in the presence of sodium acetate (4 equivalents). The acetic anhydride (4 equivalents) is then added at the temperature that is lower than the one declared in US 2019/0337896 and reacted at the same temperature for a time comprised between 1-3 hours. The use of acetonitrile in this “One-Pot” step enabled the use of acetic anhydride in the minimum quantities for having a complete reaction, hence without using it as a reaction solvent. This made its elimination by distillation simpler and therefore the use of the water/isopropanol mixture then used for the isolation of the compound of formula (I) in crude form safer.

In fact, the subsequent processing was carried out using isopropanol to separate the crude solid of the compound of formula (I).

The compound of formula (I) thus obtained in crude form is already in itself characterized by a high HPLC purity level (>90%) and the only significant impurity present is the impurity A. The compound (I) can be conveniently purified by crystallization in isopropanol/H₂O, as found surprisingly by the present Applicant, rather than according to the known methods such as the methanol/isopropanol mixture of US 2019/0337896 or acetone, isopropanol, or methanol used in the other cited references. None of the references previously mentioned the use of the isopropanol/H₂O mixture in the appropriate ratios used in the present invention, chosen from the following: Isopropanol/Water: 5.9/3.4 or 7.4/3.4 or 9.9/3.4 expressed in volumes in litres/kg of the crude compound of formula (I).

The use of isopropanol/H₂O has the advantage of providing the compound of formula (I) with purity ≥99.5 despite the fact that the intermediate (VI) has not been isolated.

As previously discussed, the Applicant prepared the compound of formula (I) as described in application US 2019/0337896, as reported in the Experimental Part, and verified that the maximum purity that can be obtained in 93%, therefore significantly lower than the claimed 99%. Such lower purity cannot be highlighted by analysing the sample at 205 nm, as described in said application.

The known impurities that this analytical method is able to effectively quantify and that the synthesis and purification process of the present invention is able to reduce to less than 0.15% are as follows:
1. Impurity A: N-phenylacetamide;
2. Impurity B: 4-(1,1-dimethyl-2-((1E,3E,5E)-6-(N-phenylacetamido)hexa-1,3,5-trienyl)-1H-benzo[e]indole-3-yl)butan-1-sulfonate;
3. Impurity C: 4-(1,1,2-trimethyl-1H-benzo[e]indole-3-yl)butan-1-sulfonate
4. Impurity D: 1-(1,1-dimethyl-2-methylidene-1,2-dihydro-3H-benzo[e]indol-3-yl)ethan-1-one.
5. Impurity E: (naphthalen-2-yl)hydrazine
6. Impurity F: N-acetyl-N′-(naphthalen-2-yl)acetohydrazide
7. Impurity G: 1,1,2,3-tetramethyl-1H-benzo[e]indol-3-ium
8. Impurity H: 4-[(2Z)-1,1-dimethyl-2-[(2E,4E,6E)-7-(1,1,3-trimethylbenzo[e]indol-3-io-2-yl)hepta-2,4,6-trienilidene]benzo[e]indol-3-yl]butan-1-sulfonate (Methyl-Indocyanine).

The impurity G was only evaluated in the compound of formula (II) as it cannot increase and its final product is in fact the impurity H.

The structures of these impurities are reported in the following diagram:

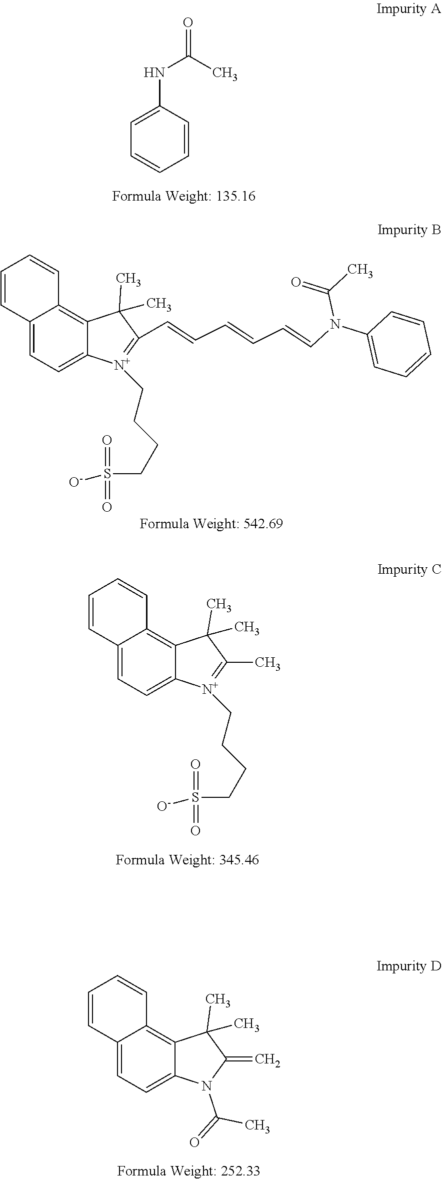

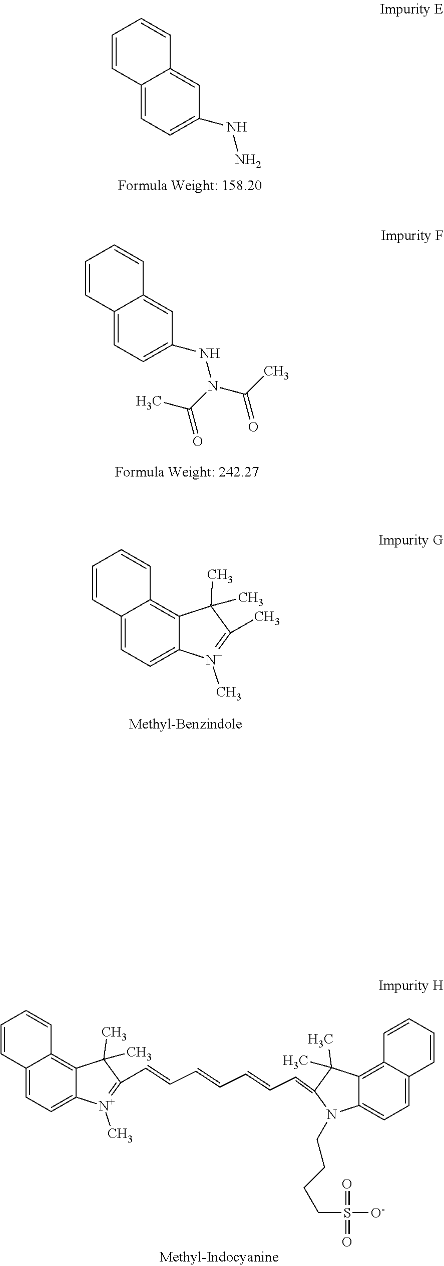 [SMILES: NNc1ccc2ccccc2c1 ; CC(=O)N(Nc1ccc2ccccc2c1)C(C)=O ; CC1=[N+](C)c2ccc3ccccc3c2C1(C)C ; C[N+]1=C(/C=C/C=C/C=C/C=C2\N(CCCCS(=O)(=O)[O-])c3ccc4ccccc4c3C2(C)C)C(C)(C)c2c1ccc1ccccc21]

The impurities E and F in particular, have warning structures as they are potentially genotoxic. The process of the present invention is able to break them down completely (≤0.05%).

The utility of the compound of formula (I) comes from its applications in diagnostics, especially for ophthalmic angiography. The compound of formula (I) is sold in the solid state in the form of sterile, freeze-dried powder containing 25 mg or 50 mg of the compound of formula (I) in the presence of no more than 5% of sodium iodide. The amount that can be administered for ophthalmic angiography must not exceed 0.1-0.3 mg/kg of body weight as a bolus injection. The 25 mg dose is dissolved in 5 ml of water for injectable solutions and the 50 mg dose in 10 ml so that the 1 ml of reconstituted injectable solution contains 5 mg of the compound of formula (I). The total daily dose in adults must be kept below 5 mg/kg of body weight.

Doses of up to 40 mg of the compound of formula (I) can be used in 2 ml of sterile water for injectable preparations. A 5 ml bolus of normal saline solution must immediately follow the injection of compound of formula (I).

The Experimental Part also describes the preparation of the freeze-dried formulation of the compound of formula (I), as obtained according to the process of the present invention, with NaI which can thus be used for preparing the bottles that are used in diagnostics.

### EXPERIMENTAL PART

### Analytical Method Used for Determining the Purity of the Compound of Formula (I)

Column HPLC: ODS Hypersil 4.6×250 mm 5 μm
Column temperature: 40° C.
Detector: UV 254 nm
Step A: Ammonium Formate 4.09 g/L at pH=5.0 with Formic Acid
Step B: Acetonitrile
Mixture phase: 70:30 A:B
Flow rate: 1.5 ml/min
Injection volume: 10 μL
Analysis time: 30 minutes
Gradient:

| 0 minutes | 70% A | 30% B |
|---|---|---|
| 14 minutes | 25% | 75% |
| 22 minutes | 25% | 75% |
| 23 minutes | 70% | 30% |
| 30 minutes | 70% | 30% |

White: mixture phase

### Sample Preparation (Method 1)

40 mg in a 50 ml flask; dissolve and bring to the correct volume with the mixture phase.

Sonicate for 5 minutes verifying complete solubilization.

### Inject the Sample Straight Away as it is Not Stable for Over 30 Minutes

### Sample Preparation (Method 2)

40 mg in a 50 ml flask; dissolve and bring to the correct volume with methanol. Sonicate for 5 minutes verifying complete solubilization.

Inject immediately.

### Inject the Sample Straight Away as it is Not Stable for Over 30 Minutes

The use of methanol enables the compound of formula (I) to be stabilized better, preventing the formation of a degradation impurity having an [MH]⁺: 752.5, i.e. “−1” with respect to the product.

Inject every 1-10 minutes.

The evaluation of potentially genotoxic impurities is obtained by injecting a larger amount of solution and the quantification is carried out as a limit assay against standard.

### Analytical Method (2) Used for Determining the Purity of the Compound of Formula (I)

Column HPLC: Polaris 3 C18-A 150×4.6 mm
Column temperature: 20° C.
Detector: UV 254 nm
Step A: Ammonium acetate 2.3 g in 1000 ml brought to pH 6.8±0.05 with diluted acetic acid or ammonia
Step B: Acetonitrile
Diluent: methanol
Flow rate: 1.5 ml/min
Injection volume: 10 μL
Analysis time: 34 minutes
Autosampler temperature: 5° C.
Gradient:

| 0 minutes | 70% A | 30% B |
|---|---|---|
| 24 minutes | 40% | 60% |
| 28 minutes | 40% | 60% |
| 29 minutes | 70% | 30% |
| 34 minutes | 70% | 30% |

Sample solution: 1.5 mg/ml in methanol. Inject immediately after preparing the solution.

This method is particularly suitable for evaluating the presence of the impurity H. It is however also able to separate all the other impurities described.

The evaluation of potentially genotoxic impurities is obtained by injecting a larger amount of solution and the quantification is carried out as a limit assay against standard.

This “analytical method 2” for ICG is LC/MS compatible and is therefore used directly in UPLC/MS (ESI⁺) with the same column so as to analyse the reaction mixtures and products also with the ESI⁺detector (Waters SQD with cone voltage: 20 volt).

This method was used to identify the impurity G and the impurity H.

### EXAMPLE 1

### Preparation of 4-(1,1,2-trimethyl-1H-benzo[e]indolyl-3-yl)butan-1-sulfonate of formula (IV)

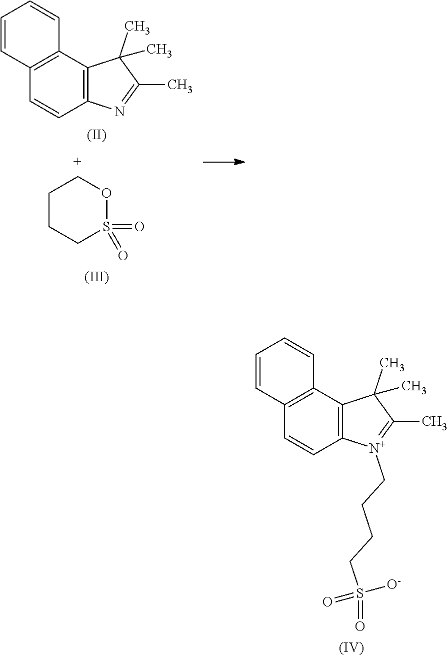

31.1 g of the compound of formula (II) (0.15 mol, 1 eq., commercially available), 40.5 g of the compound of formula (III) (0.30 mol, 2 eq., commercially available) in 93 ml of xylene are loaded into a 2 L reactor under a flow of nitrogen. The suspension is stirred and heated to the temperature of about 130° C. for 24 hours. The suspension is cooled and acetone (200 ml) is added. The solid obtained is then filtered and dried in a vacuum. Thus 48.5 g of the desired compound are obtained, corresponding to a 94.5% yield (HPLC purity: 97-98%).

Alternatively, anisol can be used as the solvent, instead of xylene, using the same amount. Following the same protocol but carrying out the reaction at 140° C., the conversion of the product is completed in 6-8 hours. The yield and quantity that can be obtained are the same as in the reaction in xylene.

### EXAMPLE 2

### Preparation of the Compound of Formula (I) (One-Pot Synthesis)

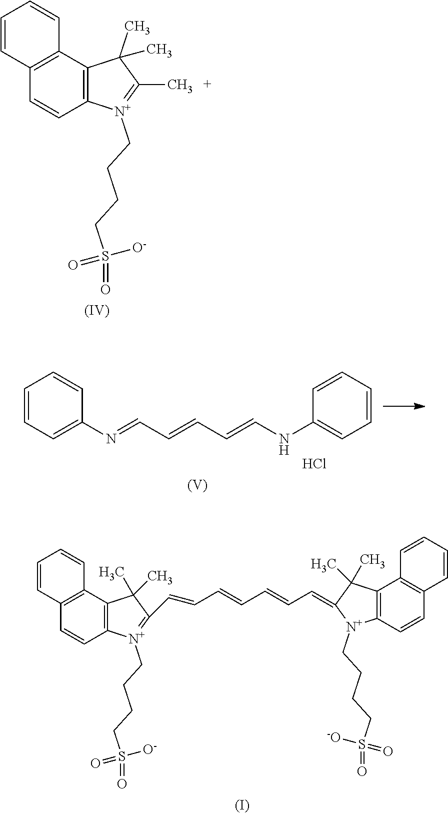

20.0 g of the compound of formula (V) (0.07 mol, 1 eq., commercially available), 48.5 g of the compound of formula (IV) (prepared as described in Example 1, 0.14 mol, 2 eq.), 23 g of sodium acetate (0.28 mol, 4 eq.) and 180 ml of acetonitrile are loaded into a 1 L reactor under a flow of nitrogen. The suspension is stirred at 20-25° C. and 28.8 g (0.28 mol, 4 eq.) of acetic anhydride are dripped in 5-10 minutes. The suspension is heated to a temperature between 45-50° C. and stirring continues for about 2 hours. The reaction mixture is concentrated in a vacuum keeping the temperature comprised between 40 and 50° C. It is then brought back to atmospheric pressure and 100 ml of iso-propanol are added then the reaction mixture is concentrated again in a vacuum, keeping the temperature comprised between 40 and 50° C.

Then water (180 ml) and isopropanol (320 ml) are loaded, the product is put into solution at 50-55° C., then isopropanol (100 ml) is added and the mixture is cooled gradually to 20-25° C.

It is filtered and washed with isopropanol.

Thus the wet desired compound is obtained (yield according to the weight loss: 46.3 g, 85.1% with respect to compound (V) with HPLC purity 80-85%.

### EXAMPLE 3

### Purification of the Compound of Formula (I) (Without NaI)

49 g of indocyanine green wet with isopropanol (prepared as described in Example 2, equal to 24.4 g dry), 130 ml of isopropanol and 77 ml of water are loaded into a 1 litre flask. It is heated to 50-55° C. and stirred until complete solubilization. The pH of the solution is corrected to 7.5-8.5 with 5% NaOH and it is cooled to 40-45° C.

Maintaining 40-45° C. isopropanol (48 ml) is added, then it is cooled gradually to 20-25° C., stirring continues for 1.5 h, then it is filtered and washed with isopropanol. (2×48 ml).

The powder is dried in a vacuum at 60° C. for 40 hours.
Yield: 20 g (82.9%)
HPLC purity: 99.5%; impurity A: 0.40%.
Sodium iodide content: 0%.

### EXAMPLE 4

### Preparation of the Compound of Formula (I) According to the Process as Described in US2019/0337896

The compound of formula (I) was prepared according to the process described in patent application US2019/0337896 starting from the intermediates (II) and (III) and (V), prepared as described in the previous examples, obtaining the desired compound of formula (I), following the synthetic sequence described in examples 1, 2, 5 and 6 of US2019/0337896. The weight yield, starting from the compound of formula (II) was 60%, whereas with the process of the present invention it is 86%.

The product obtained was then analysed with the method of the present invention by preparing the solutions of the sample both with the method 1 and with the method 2.

The purity determined with the HPLC method was equal to 93.27%.
Impurity A: 0.68%
Impurity C: 1.12%
Impurity D, E and F: non quantifiable
Maximum unknown impurity: 2.42% (rt: 0.93)

If the purity is verified at the wavelength of 205 nm as reported in the aforesaid patent it is 100%, which clearly does not correspond to reality.

### EXAMPLE 5

### Preparation of the Compound of Formula (I) with NaI (I Crystallization)

93.6 g of the crude compound of formula (I) (46.3 g of theoretical dry compound, prepared as described in Example 2) and sodium iodide (1.39 g; 3% w/w) are suspended in 250 ml of isopropanol and in 148 ml of water. The suspension is heated to a temperature of 55-60° C. and stirred until complete dissolution. The pH is corrected to 7.5-8.5 using 2.5% w/w sodium hydroxide solution. The solution is cooled to a temperature comprised between 45-50° C. and in 15-30 minutes 93 ml of isopropanol are added. It is cooled slowly until reaching the temperature of 20-25° C. and stirring continues for 30 minutes. The suspension is then brought to 35-40° C., stirred for about 1 hour, then cooled again in about 2 h to 20-25° C. and finally filtered at 20-30° C. and washed with isopropanol.

It is dried in a vacuum at 50-80° C. for 8-48 hours and 35.89 g of the desired product are obtained with a yield of 77.5% (with respect to the respective crude dry product loaded).
HPLC purity: 99.6%; impurity A: 0.28%.
Iodide (potentiometric titration with silver electrode): 1%

In the event in which the known impurities are >0.15% and the unknown ones >0.1%, it is possible to perform a second crystallization using less sodium iodide, which is essential for keeping the quantity of sodium iodide in the finished product less than 2.5% (example 6).

### EXAMPLE 6

### Preparation of the Compound of Formula (I) with NaI (II Crystallization)

The compound of formula (I), wet, obtained from the first crystallization (prepared as described in example 5) (63.7%, equal to 35.9 g of corresponding dry product based on weight loss), sodium iodide (0.54 g; 1.5% w/w), isopropanol (194 ml) and water (115 ml) are loaded into a 1 litre reactor. It is heated to 55-60° C. until complete dissolution, then the solution is filtered on cardboard and the filter is washed with water (7 ml) and then with isopropanol (18 ml). The filtrate is brought to 55-60° C., if necessary the pH is corrected with NaOH diluted in the range 7.5-8.5, then cooled to 45-50° C. and isopropanol (54 ml) is added in about 30 minutes.

It is cooled slowly to 20-25° C., heated again to 35-40° C. for about 1 hour, then brought back to 20-25° C. in about 1 hour and stirring continues for 30 minutes.

The suspension is filtered and washed with isopropanol, obtaining the wet product which is dried in a vacuum at 50-80° C. for 24-48 hours.
Yield: 26.9 g (75%).

The purity of the product thus obtained measured using the HPLC method of the invention is ≥99.5%.
HPLC purity: 99.92%
Impurity A, B, C, D, E and F: non quantifiable (HPLC);
Maximum unknown impurity: 0.084% (HPLC; rrt: 0.45).
Sodium iodide (potentiometric titration according to USP monograph): 0.9%.
Residual isopropanol: 1597 ppm.

### EXAMPLE 7

### Preparation of the Freeze-Dried Formulation of the Compound of Formula (I) as Obtained According to the Process of the Following Invention with NaI

Indocyanine green (125 mg) obtained as described in example 6 (containing NaI ≤2.5%) is dissolved in water (25 ml) and sonicated for 1 minute (5 mg/ml).

This solution is used to fill 5 different amber glass vials (about 5 ml of solution per vial). The vials are freeze dried using the following freeze drying conditions:
Freeze dryer: Edwards MINIFAST 680
Temperature: start of freeze drying −40° C.; end of freeze drying +5° C.
Pressure (Vacuum): start of freeze drying 6.6*10² mbar, end of freeze drying 4.6*10⁻² mbar
Freeze drying time: 72 h
Amber glass vials insufflated with nitrogen and sealed at the end of distillation.

### EXAMPLE 8

### Preparation of the Freeze-Dried Formulation of the Compound of Formula (I) as Obtained According to the Process of the Following Invention Without NaI

The freeze-dried formulation is prepared according to what is described in example 7, but using the compound of formula (I) prepared according to what is described in example 3. The compound of formula (I) obtained without the use of sodium iodide (sodium iodide by potentiometric titration with silver electrode=0%) is not soluble in water at 20-25° C. at the clinical use concentration of 5 mg/ml or 2.5 mg/ml if used as such, as a dry isolated powder after crystallization.

Surprisingly, the same dry-frozen powder is instead soluble at the concentrations of 2.5 mg/ml, 5 mg/ml and also 10 mg/ml.

The solubility was evaluated by filtering all the solution obtained on a syringe filter provided with 0.45 μm holes, observing that the filtration takes place fluidly, without any residue remaining either on the filter or in the vial from which the solution was withdrawn.

The molecule is probably soluble in itself, but due to kinetic reasons it does not dissolve in reasonable time scales. As the dry frozen powder, on the other hand, has a high relative surface area of contact with water, it tends to dissolve completely and immediately, without even needing to be sonicated.

### EXAMPLE 9

### Stability Tests

The stability of the powder of the compound of formula (I) obtained according to the method described in accordance with Example 6 was evaluated in the presence/absence of oxygen (i.e. air or nitrogen) not protected from light (i.e. packaged in polythene bags or in a double aluminium plus polythene bag, according to the method as described in the International patent application of the same Applicant WO2013168186) and comparing it with the stability of a commercial product containing a higher quantity of NaI (3.6% vs 1.4%).

The powder produced according to the method described is significantly more stable although having less sodium iodide, also in the absence of nitrogen, and in general characterized by greater purity also at T zero.

The results are shown in Table 1.

TABLE 1

| | | In | | | | | % rt | % rt | % rt | INNmax (excl. e) | | |
|---|---|---|---|---|---|---|---|---|---|---|---|---|
| Batch | Nal % | nitrogen | Package | Stability | t | Diluent | 3.5 (A) | 4.0 | 8.9 | % | rt | Purity |
| Commercial | 3.6 | NO | Polythene | Type | t0 | MeOH | nq | 0.12 | 0.29 | 0.08 | 9.47 | 99.28 |
| vial | | | | | | | | | | | | |

| | | | | | | | | | | | |
|---|---|---|---|---|---|---|---|---|---|---|---|
| NO | Polythene | Cell 10° | C. | 30 days | MeOH | nq | 0.24 | 0.47 | 0.47 | 8.9 | 99.05 |
| NO | Polythene | 20-25° | C. | 30 days | MeOH | nq | 0.33 | 0.45 | 0.45 | 8.9 | 98.86 |

| | | | | | | | | | | | | |
|---|---|---|---|---|---|---|---|---|---|---|---|---|
| 067/046 | 1.4 | NO | Polythene | Type | t0 | MeOH | nq | 0.02 | 0.03 | 0.07 | 11.15 | 99.73 |

| | | | | | | | | | | | |
|---|---|---|---|---|---|---|---|---|---|---|---|
| NO | Polythene | 10° | C. | 30 days | MeOH | nq | 0.04 | 0.04 | 0.06 | 11.15 | 99.84 |
| NO | Polythene | 20-25° | C. | 30 days | MeOH | nq | 0.076 | 0.05 | 0.076 | 4.0 | 99.78 |

| | | | | | | | | | | | | |
|---|---|---|---|---|---|---|---|---|---|---|---|---|
| 067/046 | 1.4 | NO | Polythene | Type | t0 | MeOH | nq | 0.02 | 0.03 | 0.07 | 11.15 | 99.73 |

| | | | | | | | | | | | |
|---|---|---|---|---|---|---|---|---|---|---|---|
| YES | Aluminium + | 10° | C. | 30 days | MeOH | nq | 0.02 | 0.04 | 0.07 | 11.25 | 99.77 |
| | Polythene | | | | | | | | | | |
| YES | Aluminium + | 20-25° | C. | 30 days | MeOH | nq | 0.03 | 0.05 | 0.08 | 11.14 | 99.71 |
| | Polythene | | | | | | | | | | |

Commercial vial: vial made of amber glass containing ICG, kept open to verify the stability in the presence of oxygen,
INNmax: unknown maximum impurity,
rt: retention time, with analytical method reported in the experimental part,
067/046: ICG batch produced as in example 6,
%: % area for internal normalization at 254 nm

### EXAMPLE 10

### A) Preparation of 4-(1,1,2-trimethyl-1H-benzo[e]indolyl-3-yl)butan-1-sulfonate of Formula (IV) in the Presence of Methanol with the Formation of the Impurity G

The preparation was carried out as described in Example 1, but in the presence of 3.3% v/v of methanol. The LC/MS analysis of this reaction highlights the presence of 0.55% of the impurity G characterized by [M]⁺: 224.18.

### B) Confirmation of the Structure of the Impurity G

To confirm the structure of the impurity G previously hypothesized in the example 10 A on the basis of the LC/MS analysis, the impurity G (Methyl-Benzindole) was synthesized according to a process known in literature (Ind. Chem. Res, 2012, 51, 3630-3638) by reacting the starting Benzindole with methyl-iodide. The product thus obtained has the expected mass spectrum, i.e. [M]⁺: 224, and co-elutes with the peak of equal weight identified in the intermediate 1, example 10 A, confirming the identity thereof.

H-NMR: compliant with the structure.

### C) Preparation of the Impurity H (Methyl-Indocyanine)

The Impurity B (prepared according to the procedure reported in U.S. Pat. No. 2,895,955, example 3; 20.0 g), methyl-benzindole iodide (14.24 g), sodium acetate (37.7 g), glacial acetic acid (23.6 ml) and acetonitrile (240 ml) are loaded into a 500 ml flask in the given order. The suspension is heated to 45/50° C. and stirring continues for six hours. The reaction mass is cooled to 20/25° C. and the reaction is stirred at 20/25° C. for another 40 hours. Glacial acetic acid (1 ml) is loaded and the reaction mixture is heated to 45/50° C. and stirring continues for five hours. The reaction mixture is cooled to 20/25° C. stirring continues for another 64 hours. Part of the solvent (about 130 ml) is distilled in a vacuum at 30/50° C. Isopropanol is loaded (40 g) and the solvent is distilled in a vacuum at 30/50° C. Water (100 ml) and isopropanol (122 g) are loaded into the reaction residue. The suspension is heated to 50/55° C. without observing complete dissolution. The suspension is maintained at 50/55° C. for 30 minutes. Isopropanol (40 ml) is loaded, maintaining the suspension at 50/55° C. It is cooled in about two hours to 30/35° C. and the suspension is stirred for an hour. The reaction mass is heated further to 40/45° C. and the suspension is stirred for another 30 minutes. The reaction mass is cooled to 20/25° C. in about an hour and it is stirred for an hour. The solid is filtered on a Buchner funnel and washed with isopropanol (2×20.0 g). The crude solid is dried at 55° C. for 16 hours.

First purification: The crude solid (23.2 g), water (74.4 g) and isopropanol (98.4 g) are loaded into a 500 ml flask in the given order. The suspension is heated to 60/65° C. without observing dissolution. The suspension is stirred at 60/65° C. for an hour. Isopropanol (36.4 g) is loaded and stirred at 60/65° C. for an hour. The reaction mass is cooled to 20/25° C. and it is stirred for five hours. The solid is filtered on a Buchner funnel and washed with isopropanol (25 ml). The solid is dried in a vacuum at 55° C. for 16 hours.

Second purification: The first dry purified product (16.9 g), water (68.3 g) and isopropanol (111.9 g) are loaded into a 250 ml flask in the given order and the suspension is heated to 60/65° C. The suspension is stirred at 60/65° C. for four hours. The suspension is cooled to 20/25° C. and is stirred at 20/25° C. for two hours. The solid is filtered on a Buchner funnel and washed with isopropanol (20 ml). The solid is dried at 50° C. in a vacuum for twenty hours. Third purification: The second purified product (14.2 g) and isopropanol (60 g) are loaded into a 250 ml flask in the given order. The suspension is heated to 70/80° C. (solvent reflux) and maintained for an hour. The reaction mass is cooled to 20/25° C. and it is stirred for five hours. The solid is filtered on a Buchner funnel and washed with isopropanol (2×15 g). The solid is dried in a stove at 50° C. for six hours and then at 60° C. for another eight hours.
Dry solid 1.57 g
(HPLC) purity: 93.07%
[MH]⁺: 631.39 (ESI⁺).

The impurity H thus prepared co-elutes with the impurity of eight m/z found in the “demo batch” mentioned in the descriptive part.

1. Method of purity determination of indocyanine green of formula (I), 1H-benz[e]indolium, 2-[7-[1,3-dihydro-1,1-dimethyl-3-(4-sulfobutyl)-2H-benz[e]indole-2-ylidene]-1,3,5-heptatrienyl]-1,1-dimethyl-3-(4-sulfobutyl) hydroxide, internal salt, sodium salt, 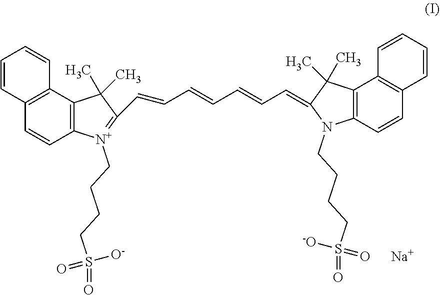
comprising the following
Column HPLC: Polaris 3 C18-A 150×4.6 mm
Column temperature: 20° C.
Detector: UV 254 nm
Step A: Ammonium acetate 2.3 g in 1000 ml brought to pH 6.8±0.05 with diluted acetic acid or ammonia
Step B: Acetonitrile
Diluent: methanol
Flow rate: 1.5 ml/min
Injection volume: 10 μL
Analysis time: 34 minutes
Autosampler temperature: 5° C.
Gradient:

| 0 minutes | 70% A | 30% B |
|---|---|---|
| 24 minutes | 40% | 60% |
| 28 minutes | 40% | 60% |
| 29 minutes | 70% | 30% |
| 34 minutes | 70% | 30% |

2. Composition comprising indocyanine green of formula (I), 1H-benz[e]indolium, 2-[7-[1,3-dihydro-1,1-dimethyl-3-(4-sulfobutyl)-2H-benz[e]indole-2-ylidene]-1,3,5-heptatrienyl]-1,1-dimethyl-3-(4-sulfobutyl) hydroxide, internal salt, sodium salt, with a total impurity content ≤0.5% and single impurity ≤0.10%, determined by the HPLC method according to claim 1 and with a NaI content ≤2.5%.

3. Process for the preparation of the compound of formula (I) as defined in claim 2, comprising the following steps:
a) reacting the compound of formula (II) 1,1,2-trimethyl-1h-benzo[e]indole, 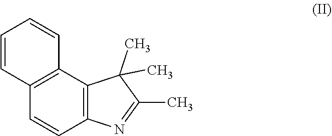 [SMILES: CC1=Nc2ccc3ccccc3c2C1(C)C]
with 1,4-butansultone of formula (III) 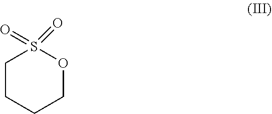
in a high-boiling solvent selected between anisole or xylene to give 4-(1,1,2-trimethyl-1H-benzo[e]indolyl-3-yl)butan-1-sulfonate of formula (IV), according to known methods; 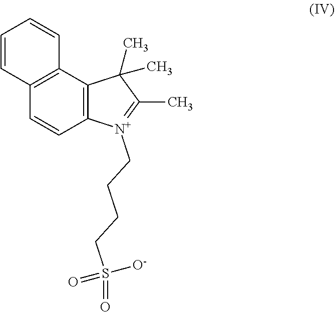 [SMILES: CC1=[N+](CCCCS(=O)(=O)[O-])c2ccc3ccccc3c2C1(C)C]
b) reacting the compound of formula (IV) with the compound of formula (V), 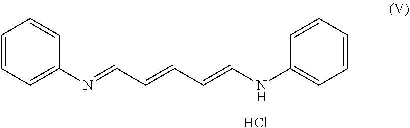 [SMILES: C(=C\C=N\c1ccccc1)/C=C/Nc1ccccc1 ; Cl]
N-phenyl-N-((1E,3E,5E)-5-(phenylammonium)penta-1,3-dienyl hydrochloride, in the presence of acetic anhydride, sodium acetate and using a dipolar aprotic solvent to give the final compound of formula (I), without isolating any intermediate.

4. Process according to claim 3, in which the dipolar aprotic solvent in step b) is acetonitrile, and the acetic anhydride and sodium acetate amount to 4 equivalents with respect to compound (IV).

5. Process according to claim 3 wherein the compound of formula (I) in crude form obtained after step b), is purified by crystallization from an isopropanol/water mixture selected from: 5.9/3.4 or 7.4/3.4 or 9.9/3.4 expressed in volumes in litres per kg of the crude compound of formula (I).

6. Composition comprising indocyanine green of formula (I) 1H-benz[e]indolium, 2-[7-[1,3-dihydro-1,1-dimethyl-3-(4-sulfobutyl)-2H-benz[e]indole-2-ylidene]-1,3,5-heptatrienyl]-1,1-dimethyl-3-(4-sulfobutyl) hydroxide, internal salt, sodium salt, with a total impurity content ≤0.5% and single impurity ≤0.10%, determined by a HPLC method comprising
Column HPLC: Polaris 3 C18-A 150×4.6 mm
Column temperature: 20° C.
Detector: UV 254 nm
Step A: Ammonium acetate 2.3 g in 1000 ml brought to pH 6.8±0.05 with diluted acetic acid or ammonia
Step B: Acetonitrile
Diluent: methanol
Flow rate: 1.5 ml/min
Injection volume: 10 μL
Analysis time: 34 minutes
Autosampler temperature: 5° C.
Gradient:
0 minutes 70% A 30% B
24 minutes 40% 60%
28 minutes 40% 60%
29 minutes 70% 30%
34 minutes 70% 30% and with a NaI content ≤2.5%. obtainable by the process according to claim 3.
